(19) United States
(12) Patent Application Publication
(10) Pub. No.: US 2021/0059990 A1
(43) Pub. Date: Mar. 4, 2021

(54) METHODS AND DRUG COMPOSITIONS FOR TREATING LYME DISEASE

(71) Applicant: The Board of Trustees of the Leland Stanford Junior University, Stanford, CA (US)

(72) Inventors: Jayakumar Rajadas, Cupertino, CA (US); Venkata Raveendra Pothineni, Mountain View, CA (US); Mohammed Inayathullah Nazir Ahmed, Santa Clara, CA (US)

(21) Appl. No.: 17/097,768

(22) Filed: Nov. 13, 2020

Related U.S. Application Data
(63) Continuation of application No. 16/070,504, filed on Jul. 16, 2018.
(60) Provisional application No. 62/279,826, filed on Jan. 17, 2016.

(51) Int. Cl. A61K 31/431; A61K 31/4545; A61K 31/546; A61K 31/5377; A61K 31/4375; A61K 31/7048; A61K 31/704; A61K 31/423; A61K 31/4709; A61P 31/04

(52) U.S. Cl. CPC A61K 31/431; A61K 31/4545; A61K 31/546; A61K 31/5377; A61K 31/4375; Y02A 50/30; A61K 31/704; A61K 31/423; A61K 31/4709; A61P 31/04; A61K 31/7048

(57) ABSTRACT

Disclosed herein are methods and drug compositions for treating Lyme disease and post-treatment Lyme disease syndrome (PTLDS) or chronic Lyme disease (CLD). In one embodiment, a method of treating a subject with Lyme disease involves administering an effective amount of a therapeutic agent selected from the group consisting of tetraethylthiuram disulfide, doxorubicin, epirubicin, azlocillin, cephalothin, josamycin, cefotaxime, cefazolin, erythromycin, calcimycin, gramicidin, cefdinir, gambogic acid, ceftazidime, ticarcillin, valinomycin, moxifloxacin, linezolid, idarubicin, tosufloxacin, loratadine, ceftriaxone, and combinations thereof, and pharmaceutical salts, hydrates, and solvates thereof.

### PRIORITY PARAGRAPH

This application claims priority to U.S. Provisional Application No. 62/279,826 filed on 17 Jan. 2016 entitled “Methods to identify compounds and compound combinations for the treatment of Borrelia burgdorferi infections” and is incorporated herein by reference.

### BACKGROUND

Lyme disease is the most common zoonotic bacterial disease in North America. More than 300,000 cases are estimated per annum in the United States (US). It is caused by the spirochetes of genus Borrelia, collectively known as B. burgdorferi sensu lato (Bsl). Among the members of the genus, B. burgdorferi sensu stricto (Bss) is the single major causative agent of the disease in the US. Other members of the genus are B. duttonii, B. garinii, B. afzelii and B. miyamotoi, B. valaisiana, B. spielmanii, B. lusitaniae, B. babesiosis and B. erlichiosis. Important clinical presentations of Lyme disease in humans include erythema migrans, fatigue, fever, chills, muscle pain and joint pain. In the absence of antibiotic therapy, B. burgdorferi (Bb) can affect immunocompetent hosts and can establish long-term infections lasting years to lifelong in mice and humans that are its natural and incidental hosts respectively.

The majority of patients with Lyme disease can be cured with the antibiotics doxycycline or amoxicillin used for 2-4 weeks, especially during the early phase of the disease. However, a subset of patients experience persistent symptoms despite antimicrobial therapy including neurocognitive difficulties, arthralgias, sleep disruption, headache, muscle and joint pain, generalized fatigue and musculoskeletal pains. When symptoms last longer than 6 months after antibiotic treatment, the condition is proposed to be post-treatment Lyme disease syndrome (PTLDS) or chronic Lyme disease (CLD).

In many cases antibiotic therapy does not fully eradicate Bb. Peristers that survive antibiotic treatment are tolerant cells and upon regrowth, they form a new persister subpopulation. Therefore, it is necessary to identify potent drug candidates that can target Borrelia that are resistant to antibiotic treatment.

### SUMMARY

Disclosed herein are therapeutic agents and methods for treating Lyme disease. In one embodiment, a method of treating a subject with Lyme disease involves administering an effective amount of a therapeutic agent selected from the group consisting of tetraethylthiuram disulfide, doxorubicin, epirubicin, azlocillin, cephalothin, josamycin, cefotaxime, cefazolin, erythromycin, calcimycin, gramicidin, cefdinir, gambogic acid, ceftazidime, ticarcillin, valinomycin, moxifloxacin, linezolid, idarubicin, tosufloxacin, loratadine, ceftriaxone, and combinations thereof, and pharmaceutical salts, hydrates, and solvates thereof. In one embodiment, the therapeutic agent is administered from about 0.1 milligram (mg)/kilogram (kg) to about 500 mg/kg body weight.

In another embodiment, a method of treating a subject having post-treatment Lyme disease syndrome (PTLDS) involves, administering an effective amount of a therapeutic agent selected from the group consisting of tetraethylthiuram disulfide, doxorubicin, epirubicin, azlocillin, cephalothin, josamycin, cefotaxime, cefazolin, erythromycin, calcimycin, gramicidin, cefdinir, gambogic acid, ceftazidime, ticarcillin, valinomycin, moxifloxacin, linezolid, idarubicin, tosufloxacin, loratadine, ceftriaxone, and combinations thereof, and pharmaceutical salts, hydrates, and solvates thereof. In one embodiment, the therapeutic agent is administered from about 0.1 mg/kg to about 500 mg/kg body weight.

In a further embodiment, a method of treating Borrelia infection in a subject involves administering an effective amount of a therapeutic agent selected from the group consisting of tetraethylthiuram disulfide, doxorubicin, epirubicin, azlocillin, cephalothin, josamycin, cefotaxime, cefazolin, erythromycin, calcimycin, gramicidin, cefdinir, gambogic acid, ceftazidime, ticarcillin, valinomycin, moxifloxacin, linezolid, idarubicin, tosufloxacin, loratadine, ceftriaxone, and combinations thereof, and pharmaceutical salts, hydrates, and solvates thereof. In one embodiment, the therapeutic agent is administered from about 0.1 mg/kg to about 500 mg/kg body weight.

In another embodiment, use of a therapeutic agent for treating Lyme disease, wherein the therapeutic agent is selected from the group consisting of tetraethylthiuram disulfide, doxorubicin, epirubicin, azlocillin, cephalothin, josamycin, cefotaxime, cefazolin, erythromycin, calcimycin, gramicidin, cefdinir, gambogic acid, ceftazidime, ticarcillin, valinomycin, moxifloxacin, linezolid, idarubicin, tosufloxacin, loratadine, ceftriaxone, and combinations thereof, and pharmaceutical salts, hydrates, and solvates thereof.

### BRIEF DESCRIPTION OF THE FIGURES

FIG. 1 depicts a Borrelia cell viability assay using agents (A) tetraethylthiuram disulfide, doxorubicin hydrochloride, and epirubicin hydrochloride and (B) azlocillin sodium and cephalothin sodium. The results represent mean+SD.

FIG. 2 depicts time kill curves for Bss isolate CA8 with (A) azlocillin sodium and (B) cefotaxime acid.

FIG. 3 depicts dose-dependent killing curve for azlocillin and cefotaxime (A) and doxycycline-resistant Borrelia assays (B).

### DETAILED DESCRIPTION

Disclosed herein are methods and compositions to treat Lyme disease in a subject. It is caused by the spirochetes of genus Borrelia, collectively, Bsl. Among the genus, Bss is the single major causative agent of the disease in the US. Other members of the genus family are B. duttonii, B. garinii, B. afzelii and B. miyamotoi, B. valaisiana, B. spielmanii, B. lusitaniae, B. babesiosis and B. erlichiosis.

The term “administering” when used in conjunction with a therapeutic means to administer a therapeutic agent to a patient whereby the therapeutic positively impacts the tissue or the organ to which it is targeted. The therapeutic agents described herein can be administered either alone or in combination (concurrently or serially) with other pharmaceuticals. For example, the therapeutic agents can be administered in combination with other vaccines, antibiotics, antiviral agents, anti-cancer or anti-neoplastic agents, or in combination with other treatment modalities such as herbal therapy, acupuncture, naturopathy, etc.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

The term “treatment” or “treating” is an intervention performed with the intention of preventing the development or altering the pathology or symptoms of a disorder. Accordingly, “treatment” can refer to therapeutic treatment or prophylactic or preventative measures. In some embodiments, the treatment is for therapeutic treatment. In some embodiments, the treatment is for prophylactic or preventative treatment. Those in need of treatment can include those already with the disorder as well as those in which the disorder is to be prevented. In some embodiments, the treatment is for experimental treatment.

The term “effective amount” as used herein generally refers to a sufficient amount of the therapeutic agent that is added to decrease, prevent or inhibit the disease. The amount will vary for each compound and upon known factors related to the item or use to which the therapeutic agent is applied.

In some embodiments, a method of treating a subject with Lyme disease involves administering an effective amount of a therapeutic agent selected from the group consisting of tetraethylthiuram disulfide, doxorubicin, epirubicin, azlocillin, cephalothin, josamycin, cefotaxime, cefazolin, erythromycin, calcimycin, gramicidin, cefdinir, gambogic acid, ceftazidime, ticarcillin, valinomycin, moxifloxacin, linezolid, idarubicin, tosufloxacin, loratadine, ceftriaxone, and combinations thereof, and pharmaceutical salts, hydrates, and solvates thereof. In some embodiments, the method involves administering a therapeutic agent selected from the group consisting of epirubicin, calcimycin, gambogic acid, valinomycin, linezolid, idarubicin, loratadine, and combinations thereof. In some embodiments, the method involves administering cefotaxime, loratadine, and combinations thereof. In some embodiments, the method involves administering azlocillin, loratadine, and combinations thereof. In some embodiments, the method involves administering cefotaxime, azlocillin, and combinations thereof. In some embodiments, the method involves administering a single therapeutic agent from any of the therapeutic agents set forth above.

In some embodiments, a method of treating a subject with Borrelia infection involves administering an effective amount of a therapeutic agent selected from the group consisting of tetraethylthiuram disulfide, doxorubicin, epirubicin, azlocillin, cephalothin, josamycin, cefotaxime, cefazolin, erythromycin, calcimycin, gramicidin, cefdinir, gambogic acid, ceftazidime, ticarcillin, valinomycin, moxifloxacin, linezolid, idarubicin, tosufloxacin, loratadine, ceftriaxone, and combinations thereof, and pharmaceutical salts, hydrates, and solvates thereof. In some embodiments, the method involves administering a therapeutic agent selected from the group consisting of epirubicin, calcimycin, gambogic acid, valinomycin, linezolid, idarubicin, loratadine, and combinations thereof. In some embodiments, the method involves administering cefotaxime, loratadine, and combinations thereof. In some embodiments, the method involves administering azlocillin, loratadine, and combinations thereof. In some embodiments, the method involves administering cefotaxime, azlocillin, and combinations thereof. In some embodiments, the method involves administering a single therapeutic agent from any of the therapeutic agents set forth above.

In some embodiments, the Borrelia infection is by Bb. In other embodiments, the bacteria comprise replicating forms of Bb, non-replicating persister forms of Bb, and combinations of replicating forms of Bb and non-replicating persister forms of Borrelia. In other embodiments, the Bb comprise a morphological form selected from the group consisting of a spirochete form, a spheroplast form, a cystic or round body form, a microcolony form, a biofilm-like and biofilm form, and combinations thereof. In some embodiments, the method involves treating Borrelia that are resistant to doxycycline, amoxicillin, or cefuroxime axetil.

In some embodiments, the method involves treating post-treatment Lyme disease syndrome (PTLDS) or chronic Lyme disease (CLD). Post-treatment Lyme disease syndrome (PTLDS) is observed in patients who were treated for Lyme disease with a recommended 1 to 4 week course of antibiotics (primary treatment) and still exhibit symptoms of fatigue, pain, or joint and muscle aches at the time they finish treatment. In some embodiments, the method involves treating a subject with PTLDS following a primary treatment of antibiotics, wherein the method involves administering an effective amount of a therapeutic agent selected from the group consisting of tetraethylthiuram disulfide, doxorubicin, epirubicin, azlocillin, cephalothin, josamycin, cefotaxime, cefazolin, erythromycin, calcimycin, gramicidin, cefdinir, gambogic acid, ceftazidime, ticarcillin, valinomycin, moxifloxacin, linezolid, idarubicin, tosufloxacin, loratadine, ceftriaxone, and combinations thereof, and pharmaceutical salts, hydrates, and solvates thereof. In some embodiments, the method involves administering a therapeutic agent selected from the group consisting of epirubicin, calcimycin, gambogic acid, valinomycin, linezolid, idarubicin, loratadine, and combinations thereof. In some embodiments, the method involves administering cefotaxime, loratadine, and combinations thereof. In some embodiments, the method involves administering azlocillin, loratadine, and combinations thereof. In some embodiments, the method involves administering cefotaxime, azlocillin, and combinations thereof. In some embodiments, the method involves administering a single therapeutic agent from any of the therapeutic agents set forth above.

In the methods disclosed herein, one or more therapeutic agents can be administered. For example, a first therapeutic agent can be administered with a second therapeutic agent concomitantly or subsequently. In some embodiments, there might be a lag period of few hours to days between administration of the first therapeutic agent and the second therapeutic agent.

In some embodiments, one or more therapeutic agents are administered at a dose of about 0.1 mg/kg to about 500 mg/kg body weight, about 0.1 mg/kg to about 250 mg/kg body weight, about 0.1 mg/kg to about 100 mg/kg body weight, about 0.1 mg/kg to about 50 mg/kg body weight, about 0.1 mg/kg to about 10 mg/kg body weight, or about 0.1 mg/kg to about 5 mg/kg body weight, preferably 1 mg/kg to about 2 mg/kg body weight.

In some embodiments, the therapeutic agent is administered for a duration of about 5 days to 365 days, about 5 days to 300 days, about 5 days to 300 days, about 5 days to 250 days, about 5 days to 200 days, about 5 days to 100 days, about 5 days to 60 days, about 5 days to 30 days, about 5 days to 14 days, or about 3 days to 7 days, preferably about 21 days to 28 days.

The therapeutic agents may be administered once daily or multiple times in a day. For example, a treatment regimen may include administering azlocillin 10 mg/kg twice daily, 20 mg/kg twice daily, 50 mg/kg once daily, 10 mg/kg three times daily, 20 mg/kg four times daily, or 50 mg/kg twice daily.

In some embodiments, the treatment regimen may include administering loratadine 20 mg/kg four times daily, 30 mg/kg four times daily, 20 mg/kg two times daily, or 20 mg/kg three times daily.

In some embodiments, the treatment regimen may include administering loratadine 20 mg/kg four times daily, cefotaxime 25 mg/kg once daily, and azlocillin 10 mg/kg twice daily for ten days.

In some embodiments, the treatment regimen may include administering cefotaxime 25 mg/kg once daily and azlocillin 10 mg/kg twice daily for fifteen days.

In some embodiments, the treatment regimen may include administering loratadine 20 mg/kg four times daily, preferably 100 microgram (μg)/kg to 400 μg/kg and cefotaxime 25 mg/kg once daily for ten days.

In some embodiments, the therapeutic agent disclosed herein is administered in combination with a vaccine against Borrelia species. Of particular interest are vaccines, which stimulate the production of a humoral immune response to Bb in humans. The antigens comprising the vaccine preparation may be native or recombinantly produced toxin proteins from Bb, as well as other species (e.g, B. garinii, and B. afzelii).

In some embodiments, the therapeutic agents may be present in pharmaceutical compositions or formulations that are adapted to deliver a prescribed dosage of one or more therapeutic agents to a cell, a group of cells, an organ or tissue, an animal or a human. Methods of incorporating therapeutic agents into pharmaceutical preparations are widely known in the art. The determination of an appropriate prescribed dosage of a pharmacologically active compound to include in a pharmaceutical composition in order to achieve a desired biological outcome is within the skill level of an ordinary practitioner of the art. The pharmaceutical compositions may include excipients, such as without limitation, binders, coating, disintegrants, fillers, diluents, flavors, colors, lubricants, glidants, preservatives, sorbents, sweeteners, conjugated linoleic acid (CLA), gelatin, beeswax, purified water, glycerol, any type of oil, including, without limitation, fish oil or soybean oil, or the like. Pharmaceutical compositions can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols. It will further be appreciated by an ordinary practitioner of the art that the term also encompasses those pharmaceutical compositions that contain an admixture of two or more pharmacologically active compounds, such compounds being administered, for example, as a combination therapy.

A pharmaceutical composition may be provided as sustained-release or timed-release formulations. Such formulations may release a bolus of a compound from the formulation at a desired time, or may ensure a relatively constant amount of the compound present in the dosage is released over a given period of time. Terms such as “sustained release,” “controlled release,” or “timed release” and the like are widely used in the pharmaceutical arts and are readily understood by a practitioner of ordinary skill in the art.

Pharmaceutical compositions containing the therapeutic agents described herein are typically administered orally but any suitable route of administration may be employed for providing a subject with an effective dosage of drugs of the chemical compositions described herein. For example, oral, rectal, topical, parenteral, ocular, pulmonary, nasal, and the like may be employed. Dosage forms include tablets, troches, dispersions, suspensions, solutions, capsules, creams, ointments, aerosols, and the like. In certain embodiments, it may be advantageous that the compositions described herein be administered orally.

The pharmaceutical compositions of the therapeutic agent disclosed herein can be administered in the conventional manner by any route where they are active. Administration can be systemic, parenteral, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration of the composition of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

For administration by inhalation or intranasal, the drugs used in the present invention are conveniently delivered in the form of an aerosol spray presentation from pressurized packs or nebulizers. The compounds may also be delivered in the form of a cream, liquid, spray, powder, or suppository. A metered dose of the formulation can be provided from a reservoir of the formulation. In addition, predetermined dosages can be provided, for example, suppository forms can be provided for insertion into the nose having a predetermined dosage. Kits can be provided, where prepared dosage forms and instructions for administering the dosages are included.

Suitable topical formulations for use in the present embodiments may include transdermal devices, aerosols, creams, ointments, lotions, dusting powders, gels, and the like.

In practical use, therapeutic agents used can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

The pharmaceutical compositions may be manufactured in a manner which is itself known to one skilled in the art, for example, by means of conventional mixing, granulating, dragee-making, softgel encapsulation, dissolving, extracting, or lyophilizing processes. Thus, pharmaceutical compositions for oral use may be obtained by combining the active compounds with solid and semi-solid excipients and suitable preservatives. Optionally, the resulting mixture may be ground and processed. The resulting mixture of granules may be used, after adding suitable auxiliaries, if desired or necessary, to obtain tablets, softgels, lozenges, capsules, or dragee cores.

The therapeutic agents are typically administered in admixture with suitable pharmaceutical diluents, excipients, or carriers (collectively referred to herein as “pharmacologically inert carriers”) suitably selected with respect to the intended form of administration, that is, oral tablets, capsules, elixirs, syrups and the like, and consistent with conventional pharmaceutical practices.

Suitable excipients may be fillers such as saccharides (e.g., lactose, sucrose, or mannose), sugar alcohols (e.g., mannitol or sorbitol), cellulose preparations and/or calcium phosphates (e.g., tricalcium phosphate or calcium hydrogen phosphate). In addition binders may be used such as starch paste (e.g., maize or corn starch, wheat starch, rice starch, potato starch, gelatin, tragacanth, methyl cellulose, hydroxypropylmethylcellulose, sodium carboxymethylcellulose, and/or polyvinyl pyrrolidone). Disintegrating agents may be added (e.g., the above-mentioned starches) as well as carboxymethyl-starch, cross-linked polyvinyl pyrrolidone, agar, or alginic acid or a salt thereof (e.g., sodium alginate) Auxiliaries are, above all, flow-regulating agents and lubricants (e.g., silica, talc, stearic acid or salts thereof, such as magnesium stearate or calcium stearate, and/or polyethylene glycol, or PEG). Dragee cores are provided with suitable coatings, which, if desired, are resistant to gastric juices. Softgelatin capsules (“softgels”) are provided with suitable coatings, which, typically, contain gelatin and/or suitable edible dye(s). Animal component-free and kosher gelatin capsules may be particularly suitable for the embodiments described herein for wide availability of usage and consumption. For this purpose, concentrated saccharide solutions may be used, which may optionally contain gum arabic, talc, polyvinyl pyrrolidone, polyethylene glycol (PEG) and/or titanium dioxide, lacquer solutions and suitable organic solvents or solvent mixtures, including dimethylsulfoxide (DMSO), tetrahydrofuran (THF), acetone, ethanol, or other suitable solvents and co-solvents. In order to produce coatings resistant to gastric juices, solutions of suitable cellulose preparations such as acetylcellulose phthalate or hydroxypropylmethyl-cellulose phthalate, may be used. Dye stuffs or pigments may be added to the tablets or dragee coatings or softgelatin capsules, for example, for identification or in order to characterize combinations of active compound doses, or to disguise the capsule contents for usage in clinical or other studies.

Other pharmaceutical compositions that may be used orally include push-fit capsules made of gelatin, as well as soft, thermally sealed capsules made of gelatin and a plasticizer such as glycerol or sorbitol. The push-fit capsules may contain the active compounds in the form of granules that may be mixed with fillers such as, for example, lactose, binders such as starches, and/or lubricants such as talc or magnesium stearate and, optionally, stabilizers and/or preservatives. In soft capsules, the active compounds may be dissolved or suspended in suitable liquids, such as fatty oils such as rice bran oil or peanut oil or palm oil, or liquid paraffin. In some embodiments, stabilizers and preservatives may be added.

Liquid dosage forms for oral administration can contain coloring and flavoring to increase subject acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable formulations for parenteral administration include, but are not limited to, suspensions of the active compounds as appropriate oily injection suspensions may be administered, particularly suitable for intramuscular injection. Suitable lipophilic solvents, co-solvents (such as DMSO or ethanol), and/or vehicles including fatty oils, for example, rice bran oil or peanut oil and/or palm oil, or synthetic fatty acid esters, for example, ethyl oleate or triglycerides, may be used. Liposomal formulations, in which mixtures of the chemical compositions described herein with, for example, egg yolk phosphotidylcholine (E-PC), may be made for injection. Optionally, the suspension may contain stabilizers, for example, antioxidants such as BHT, and/or preservatives, such as benzyl alcohol.

### Example 1: High-Throughput Primary Screening of Chemical Libraries with Bb Persisters to Identify Potent Drugs

### Bacterial Strains and Culture

Bss strains CA4 and CA8 originated biologically from I. pacificus ticks, United States. These strains are infectious low passage numbers, which were cultured in Barbour-Stoenner-Kelly II (BSK-II) complete medium, with 6% rabbit serum (Sigma, St. Louis, MO, USA). The cultures were grown in sterile 50 mL Falcon tubes (Corning, N.Y., USA) and incubated at 33° C. All culture media were sterilized with 0.2 μM filter units (Millipore, Billerica, Mass., USA). The Bb cultures were grown for 7-10 days to reach the stationary phase with cell density more than 10⁸/ml for performing all the assays. For HTS drug screening, 7-10 day-old stationary-phase Bb cultures were transferred to 384 well culture microplates.

### Drugs and Drug Libraries

All the information regarding purchase, solubility and stock solutions of drugs used in this study have been provided in the Table 1. All the drug stocks were filter sterilized with 0.2 μM filter units. The FDA-approved drug libraries, the Library of Pharmacologically Active Compounds (LOPAC1280), the NIH Clinical Collection (NIHCC), the Microsource Spectrum, and the Biomol FDA (now Enzo Life Sciences) were acquired from High-Throughput Bioscience Center (HTBC), Stanford University. All the library stocks were maintained in dimethyl sulfoxide (DMSO) solutions at 10 mM compound concentrations. Plate-to-plate dilutions were performed in 384-well plates by using an Evolution P3 system equipped with a 384-well head.

High-Throughput Screening of Chemical Libraries with Bb Persisters

To identify the effect of chemical compounds on Bb stationary phase cultures qualitatively, HTS screening was performed using the following procedure. 50 μl of BSK-II medium was added to white 384-well coming plates (Corning, N.Y., USA) using the Matrix Wellmate and ˜100 nL of each compound from the stock solution was added using the pin tool in the Staccato System (CaliperLS) to 1 to 22 columns. The last 2 columns of the 384 wells were left for culturing the controls. One compound per well was screened in a 384-well microplate format. These are screened in a 7-point titration ranging from 25 μM, 12.5 μM, 7.25 μM, 3.625 μM, 1.81 μM, 0.9 μM, 0.45 μM. To these plates 25 μl of 10⁶/ml Bb stationary phase cultures was added using the Multidrop dispenser. Then the plates were incubated at 33° C. for 96 hours in a humidified CO2 incubator (Forma Scientific, Massachusetts, USA) having 5% CO₂ and 95% air. After 96 hours, 25 μl of BacTiter Glo reagent (Promega, USA) was added to the plates by using Multidrop dispenser. The plates were shaken for 2 minutes and incubated at room temperature for 5 min. Finally, the luminescence was measured on a Flexstation 3 (Molecular Devices) (500 ms/well). The data was analyzed with Assay Explorer software. Hits were identified as compounds that resulted in a decrease in the luciferase signal compared to controls with no compound.

### Determination of Minimum Inhibitory Concentration (MIC) and Minimum Bactericidal Concentration (MBC)

The Minimum Inhibitory Concentration (MIC) of the small molecules identified through screening was determined by culturing 10⁶/ml Borrelia in BSK-II medium with different concentrations (0.3 μM to 160 μM) of drugs. For MIC, 1 ml cultures with respective drugs were grown in 48 well plates in triplicates, wrapped with paraffin film and placed for 72 hours at 33° C. in a humidified CO2 incubator (Forma Scientific, USA) having 5% CO2 and 95% air. Cell proliferation was assessed using bacterial counting chamber (Petroff-Hausser Counter, Horsham, Pa., USA) by phase contrast microscopy. In parallel cell proliferation was also assessed BacTiter-Glo™ Assay. The counting was performed in all the 25 squares of the central grid. The BacTiter-Glo™ Assay was performed by mixing 100 μl of culture in each well with 100 μl of BacTiter-Glo™ reagent (Promega, Madison, Wis., USA) Then, the assay was performed according to the manufacturer's instructions. Luminescence was measured on a Flex station3 micro-plate reader at luminescence 500 ms.

For determining minimum bactericidal concentration (MBC), 20 μl of the 10⁶/ml Borrelia cultures grown in BSK-II medium for 72 hours at different drug concentrations were added to the fresh BSK-II medium and sub-cultured for 3 weeks. After 3 weeks of incubation period, the samples were observed microscopically for motile spirochetes in the culture. The MIC and MBC determinations were done thrice independently.

### Time-Kill Studies

Time-kill studies were performed with Borrelia isolate CA8 Bss with azlocillin sodium and cefotaxime acid. To determine the rate of antimicrobial activity, 10⁶/ml Borrelia cultures were grown in BSK-II medium with drugs at different concentrations. BSK-II medium with no drugs is used as a control. The antibacterial activity was determined by counting bacteria at 24 hours, 48 hours and 72 hours. The experiment was done once with triplicates.

### Dose-Dependent Killing of Bb Drug-Tolerant Persisters by Drugs

For evaluating whether persisters were killed, 10⁶/ml of stationary phase Borrelia culture were tested at different concentrations (1 μM to 80 μM) of proposed lead drugs in 48-well plates in triplicate. The plates were covered and incubated for 5 days at 33° C. Pellets were extracted by centrifugation, re-suspended in fresh BSKII medium and cultured on semisolid BSK-II agarose plates for minimum 21 days. The plates were incubated for regrowth and colonies were counted. The dose-dependent killing studies were performed thrice independently.

Efficacy of the Proposed Molecules were Determined In Vivo

After the synergy was determined the efficacy of the drugs were studied in vivo in the following way: C3H/HeN mice of 4-6 week old were infected intradermally in the abdominal area with 0.1 ml of BSK-II medium containing 10⁵ Bb with a 25 G tuberculin syringe. After 7 days of infection, the mice were given a single daily dose of the lead molecules (varying mg/kg of body weight depending on the drug, or the excipient diluent control (placebo) for 5 consecutive days. The drugs were administered by intraperitoneal injection. This duration of 5 days treatment with drugs is enough as most of the researchers were following this method. The drugs that can effectively clear the Borrelia infection in 5 days have potential to use in humans in future. Two days after the last dose of lead molecule or placebo, the mice were euthanized and cultures of their skin, heart and joint tissues were collected and transferred into fresh BSK-II medium. Cultures were monitored up to 6 weeks for motile spirochetes using darkfield microscopy. The Table 2 contains the information of drugs used in the study, concentration tested in mice and the number of mice used in the study.

### Results

To identify safe and effective molecules for Lyme disease treatment, 4366 chemical compounds representing different libraries including: Sigma LOFAC (1280 compounds), NIH Clinical Collection (446 compounds), Microsource Spectrum (2000 compounds), Biomol FDA (640 compounds) were screened. The screening was repeated 7 times with a titration range from 0.45 μM to 25 μM to confirm the reproducibility, meaning, each drug was tested at different concentrations in 7 different plates. By successfully screening these libraries, nearly 150 hit molecules that showed more than 90% inhibition of bacterial growth compared to the control were identified. Out of these 150 hit molecules, 101 (67.3%) small molecules were FDA-approved compounds.

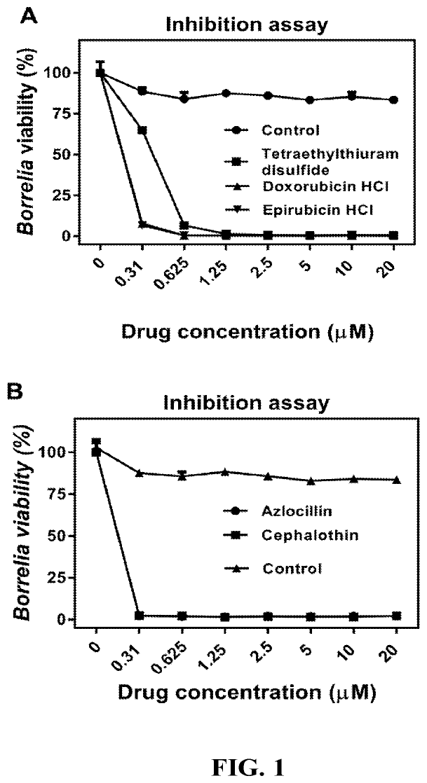

Based on the results from the primary screening, the top 20 candidates were chosen (Table 1) and reconfirmed by secondary screening with BacTiter-Glo™ Assay. These candidates were chosen based on more than 95% inhibition of bacteria in the primary screening. The inhibition by these compounds was greater than doxycycline, which is one of the currently prescribed drugs for Lyme disease. The viability of Borellia cultures was evaluated with BacTiter-Glo™ Assay. As shown in the FIG. 1A, tetraethylthiuram disulfide showed complete Borrelia cell inhibition at 1.25 μM, while epirubicin hydrochloride and doxorubicin hydrochloride were uniformly effective against Bb at 0.625 μM. Vehicle control (DMSO), did not show any significant effect on the cell survival. Drugs azlocillin and cephalothin showed nearly 99% efficacy at 0.31 μM (FIG. 1B).

TABLE 1

| S. No | Name | Supplier | Solubility | Stock solution |
|---|---|---|---|---|
| 1 | Tetraethylthiuram disulfide | Cayman chemical | DMSO | 10 mM |
| 2 | Doxorubicin hydrochloride | Cayman chemical | Water | 10 mM |
| 3 | Josamycin | Sigma-Aldrich | Ethanol | 20 mM |
| 4 | Cefotaxime acid | Cayman chemical | DMSO | 20 mM |
| 5 | Cefazolin sodium | Cayman chemical | Water | 10 mM |
| 6 | Epirubicin hydrochloride | Cayman chemical | Water | 10 mM |
| 7 | Erythromycin ethylsuccinate | Santa Cruz biotech | DMSO | 20 mM |
| 8 | A-23187 calcimycin | Cayman chemical | DMSO | 10 mM |
| 9 | Gramicidin | Sigma-aldrich | DMSO | 20 mM |
| 10 | Cefdinir | Sigma-aldrich | DMSO | 20 mM |
| 11 | Gambogic Acid | Cayman chemical | DMSO | 20 mM |
| 12 | Cephalothin sodium | Santa Cruz biotech | Water | 10 mM |
| 13 | Ceftazidime | Cayman chemical | DMSO | 5 mM |
| 14 | Ticarcillin disodium | Santa Cruz biotech | Water | 20 mM |
| 15 | Valinomycin | Cayman chemical | Ethanol | 20 mM |
| 16 | Moxifloxacin hydrochloride | Santa Cruz biotech | Water | 20 mM |
| 17 | Linezolide | Santa Cruz biotech | Water | 15 mM |
| 18 | Idarubicin HCl | Sigma-Aldrich | Water | 20 mM |
| 19 | Tosufloxacin tosylate | Santa Cruz biotech | DMSO | 10 mM |
| 20 | Azlocillin sodium | Santa Cruz biotech | Water | 10 mM |

### Determinations of MIC and MBC Values

To confirm the efficacy of the screening MIC and MBC values were evaluated. The MIC is determined as the lowest concentration at which no motile spirochete is observed by microscopy. Of the top 20 candidates evaluated, the MIC values of epirubicin hydrochloride and doxorubicin hydrochloride were less than 1 μM. For the drugs cefazolin sodium, gramicidin, azlocillin sodium, cefotaxime, cephalothin sodium, tetraethylthiuram disulfide, and linezolid, the MIC values were ≤ to 3 μM. The remaining drugs exhibited MIC values of ≥3 μM. The MBC was determined by sub-culturing 20 μl of the Borrelia cultures grown at different drug concentrations in fresh BSK-II medium for 21 days. The MBC value was determined when no spirochete was observed microscopically in the culture. Of the top 20 compounds the lowest MBC values were observed for epirubicin hydrochloride, and doxorubicin hydrochloride, which has less than 1.5 μM. The drugs gramicidin, azlocillin sodium, leucomycin, cefotaxime, idarubicin, and tetraethylthiuram disulfide exhibited MBC values under or equal to 10 μM. All the remaining drugs exhibited MBC values higher than 10 μM.

### Time Kill Studies

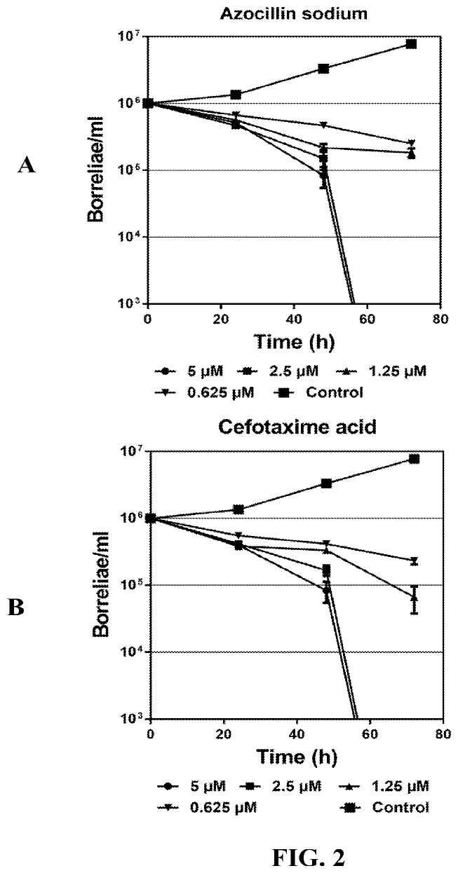

To determine the rate of antimicrobial activity of azlocillin sodium and cefotaxime acid, CA8 strain of Borrelia was exposed to different concentrations (0.625 μM, 1.25 μM, 2.5 μM and 5 μM) of each of the drugs. The initial Borrelia inoculum did not decrease 1-log 10-unit at concentrations 0.625 μM and 1.25 μM for azlocillin sodium (FIG. 2A). For cefotaxime acid at concentration 1.25 μM, the microbial growth is decreased 1-log 10-unit but not at 0.625 μM (FIG. 2B). Both azlocillin sodium and cefotaxime acid reduced morphologically intact motile cells to 3-log 10-unit (99.9%) between 48 and 72 h at concentrations 2.5 μM and 5 μM.

### Killing of Bb Persisters by Azlocillin and Cefotaxime

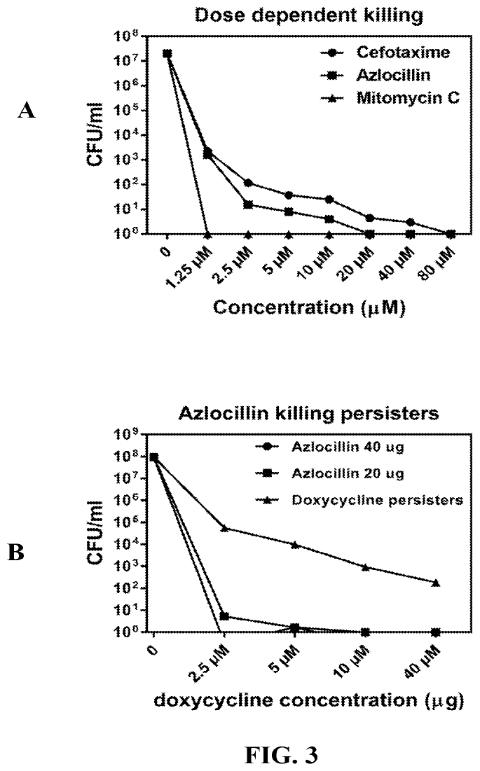

Preliminary dose-dependent killing assay for Borrelia persisters showed that azlocillin and cefotaxime eradicated drug-tolerant persisters completely at 20 μM (10 μg) concentration and 80 μM (40 μg) concentration respectively (FIG. 3A).

### Studying Efficacy of the Molecules In Vivo

To evaluate the efficacy of the drugs in vivo, they were tested in a C3H mouse model. Azlocillin, Loratadine, Cefotaxime and their combinations were tested in the C3H mice model. The results are shown in Table 2. Cefotaxime acid+Azlocillin sodium, Cefotaxime acid+Loratadine, Loratadine+Azlocillin sodium, and Cefotaxime and Azlocillin alone were very effective in eliminating Borrelia infection, based on the failure to culture live spirochetes from the urinary bladders, heart, and ear skin after treatment. Live spirochetes were observed under dark field microscopy in control saline.

TABLE 2
In vivo efficacy of drugs against Bb in C3H mice

| | | Number | Number | Number of mice |
|---|---|---|---|---|
| | | of mice | of mice | cured/Number |
| Drug | Dose | infected | treated | of mice given |
| Azlocillin | 50 mg/kg | 7 | 7 | 7/7 |
| Loratadine + Azlocillin | 1 mg/kg + 10 mg/kg | 4 | 4 | 4/4 |
| Cefotaxime + Loratadine | 30 mg/kg + 10 mg/kg | 4 | 4 | 4/4 |
| Cefotaxime | 30 mg/kg | 6 | 6 | 6/6 |
| Cefotaxime + Azlocillin | 20 mg/kg + 30 mg/kg | 4 | 4 | 4/4 |
| Ceftriaxone | 30 mg/kg | 3 | 3 | 3/3 |
| Saline (control) | | 6 | 6 | 0/6 |

### Eradication of Doxycycline-Tolerant Bb by Azlocillin

It is proved that Borrelia persisters survive the antibiotic challenge of currently prescribed drugs like doxycycline, amoxicillin and ceftriaxone. So to identify the drugs that kill drug-tolerant Borrelia persisters, 10⁶/ml Borrelia in BSK-II medium was treated with concentrations of doxycycline ranging from 2.5 μM to 40 μM for 3 days. The doxycycline-tolerant persisters were treated with 20 μg and 40 μg Azlocillin for 5 days. After 5 days of treatment, the cultures were pelleted, washed and plated on semisolid BSK-II agarose plates for minimum 21 days. The plates were incubated for regrowth and colonies are counted. The study was performed thrice independently. At concentrations 20 μg and 40 μg, azlocillin completely eliminated drug-tolerant Borrelia persisters, which are shown in FIG. 3B. 40 μg of cefotaxime killed the Borrelia persisters completely.

Normally, ClpP, an intracellular protease recognizes and eliminates misfolded proteins with the aid of ATP-dependent ClpX, C or A subunits. 30-fold upregulation of clpP encoding ATP-dependent Clp protease proteolytic subunit is observed in doxycycline-tolerant Bb persisters. Doxycycline could disturb bacterial protein synthesis by binding to the 30S ribosomal subunit that might lead to misfolded proteins. The up-regulation of clpP could be a response to this. In this adverse process, Borrelia might shut down its protein synthesis and turn into persisters. We propose that Azlocillin binds to ClpP and keeps the catalytic chamber open, allowing entry to peptides and proteins. Therefore, azlocillin targets growing cells with active protein synthesis and can eradicate Borrelia.

What is claimed is:

1. A method of treating a subject having Lyme disease comprising administering an effective amount of a therapeutic agent selected from the group consisting of tetraethylthiuram disulfide, doxorubicin, epirubicin, azlocillin, cephalothin, josamycin, cefotaxime, cefazolin, erythromycin, calcimycin, gramicidin, cefdinir, gambogic acid, ceftazidime, ticarcillin, valinomycin, moxifloxacin, linezolid, idarubicin, tosufloxacin, loratadine, ceftriaxone, and combinations thereof, and pharmaceutical salts, hydrates, and solvates thereof.

2. The method of claim 1, wherein the therapeutic agent is selected from the group consisting of epirubicin, calcimycin, gambogic acid, valinomycin, linezolid, idarubicin, loratadine, and combinations thereof.

3. The method of claim 1, wherein the therapeutic agent is selected from the group consisting of azlocillin, loratadine, and combinations thereof.

4. The method of claim 1, wherein the therapeutic agent is selected from the group consisting of cefotaxime, loratadine, and combinations thereof.

5. The method of claim 1, wherein the therapeutic agent is selected from the group consisting of cefotaxime, azlocillin, and combinations thereof.

6. The method of claim 1, wherein the administration is parenteral, subcutaneous, intravenous, intramuscular, intraperitoneal, transdermal, oral, buccal, ocular, vaginal, nasal, topical, and combinations thereof.

7. The method of claim 1, wherein the therapeutic agent is administered from about 0.1 milligram/kilogram to about 500 milligram/kilogram body weight.

8. The method of claim 1, wherein the subject has post-treatment Lyme disease syndrome (PTLDS).

9. A method of treating a Borrelia infection in a subject comprising administering an effective amount of a therapeutic agent selected from the group consisting of tetraethylthiuram disulfide, doxorubicin, epirubicin, azlocillin, cephalothin, josamycin, cefotaxime, cefazolin, erythromycin, calcimycin, gramicidin, cefdinir, gambogic acid, ceftazidime, ticarcillin, valinomycin, moxifloxacin, linezolid, idarubicin, tosufloxacin, loratadine, ceftriaxone, and combinations thereof, and pharmaceutical salts, hydrates, and solvates thereof.

10. The method of claim 9, wherein the administration is parenteral, subcutaneous, intravenous, intramuscular, intraperitoneal, transdermal, oral, buccal, ocular, vaginal, nasal, topical, and combinations thereof.

11. The method of claim 9, wherein the therapeutic agent is administered from about 0.1 milligram/kilogram to about 500 milligram/kilogram body weight.

12. The method of claim 9, wherein the therapeutic agent is selected from the group consisting of epirubicin, calcimycin, gambogic acid, valinomycin, linezolid, idarubicin, loratadine, and combinations thereof.

13. The method of claim 9, wherein the therapeutic agent is selected from the group consisting of azlocillin, loratadine, and combinations thereof.

14. The method of claim 9, wherein the therapeutic agent is selected from the group consisting of cefotaxime, loratadine, and combinations thereof.

15. The method of claim 9, wherein the therapeutic agent is selected from the group consisting of cefotaxime, azlocillin, and combinations thereof.

16. A method of treating a subject having post-treatment Lyme disease syndrome (PTLDS) comprising administering an effective amount of a therapeutic agent selected from the group consisting of tetraethylthiuram disulfide, doxorubicin, epirubicin, azlocillin, cephalothin, josamycin, cefotaxime, cefazolin, erythromycin, calcimycin, gramicidin, cefdinir, gambogic acid, ceftazidime, ticarcillin, valinomycin, moxifloxacin, linezolid, idarubicin, tosufloxacin, loratadine, ceftriaxone, and combinations thereof, and pharmaceutical salts, hydrates, and solvates thereof.

17. The method of claim 16, wherein the administration is parenteral, subcutaneous, intravenous, intramuscular, intraperitoneal, transdermal, oral, buccal, ocular, vaginal, nasal, topical, and combinations thereof.

18. The method of claim 16, wherein the therapeutic agent is administered from about 0.1 milligram/kilogram to about 500 milligram/kilogram body weight.

19. The method of claim 16, wherein the therapeutic agent is selected from the group consisting of epirubicin, calcimycin, gambogic acid, valinomycin, linezolid, idarubicin, loratadine, and combinations thereof.

20. Use of a therapeutic agent for treating Lyme disease, wherein the therapeutic agent is selected from the group consisting of tetraethylthiuram disulfide, doxorubicin, epirubicin, azlocillin, cephalothin, josamycin, cefotaxime, cefazolin, erythromycin, calcimycin, gramicidin, cefdinir, gambogic acid, ceftazidime, ticarcillin, valinomycin, moxifloxacin, linezolid, idarubicin, tosufloxacin, loratadine, ceftriaxone, and combinations thereof, and pharmaceutical salts, hydrates, and solvates thereof.

21. Use of a therapeutic agent for treating post-treatment Lyme disease syndrome (PTLDS), wherein the therapeutic agent is selected from the group consisting of tetraethylthiuram disulfide, doxorubicin, epirubicin, azlocillin, cephalothin, josamycin, cefotaxime, cefazolin, erythromycin, calcimycin, gramicidin, cefdinir, gambogic acid, ceftazidime, ticarcillin, valinomycin, moxifloxacin, linezolid, idarubicin, tosufloxacin, loratadine, ceftriaxone, and combinations thereof, and pharmaceutical salts, hydrates, and solvates thereof.

22. Use of a therapeutic agent for treating Borrelia infection, wherein the therapeutic agent is selected from the group consisting of tetraethylthiuram disulfide, doxorubicin, epirubicin, azlocillin, cephalothin, josamycin, cefotaxime, cefazolin, erythromycin, calcimycin, gramicidin, cefdinir, gambogic acid, ceftazidime, ticarcillin, valinomycin, moxifloxacin, linezolid, idarubicin, tosufloxacin, loratadine, ceftriaxone, and combinations thereof, and pharmaceutical salts, hydrates, and solvates thereof.
